(19) United States
(12) Patent Application Publication
(10) Pub. No.: US 2016/0166228 A1
(43) Pub. Date: Jun. 16, 2016

(54) ROTATION SPEED CORRECTION FOR CT SCANNER SYSTEM

(71) Applicant: SHENYANG NEUSOFT MEDICAL SYSTEMS CO., LTD., Shenyang (CN)

(72) Inventor: Xinliang Li, Shenyang (CN)

(21) Appl. No.: 14/965,840

(22) Filed: Dec. 10, 2015

(30) Foreign Application Priority Data
Dec. 16, 2014 (CN) 201410784812.X
Dec. 4, 2015 (CN) 201510886210.X

(51) Int. Cl. A61B 6/00; G05B 15/02

(52) U.S. Cl. CPC A61B 6/582; G05B 15/02

(57) ABSTRACT

A method for correcting rotation speed of a rotatable gantry in a CT scanner system is provided. The method may include: detecting an actual rotation speed of the rotatable gantry; determining a deviation percentage of the actual rotation speed from an instructed rotation speed sent to the rotatable gantry; and if the deviation percentage is smaller than a first preset threshold and larger than a second preset threshold, correcting the instructed rotation speed according to the deviation percentage.

### CROSS-REFERENCE TO RELATED APPLICATIONS

The present application claims priority to Chinese Patent Application No. 201410784812.X, filed on Dec. 16, 2014, and Chinese Patent Application No. 201510886210.X, filed on Dec. 4, 2015, the entire contents of which are hereby incorporated by reference for all purposes.

### BACKGROUND

The present disclosure relates to the technical field of medical equipment.

For the rotation part in a Computer Tomography (CT) scanner system, a variable frequency motor or servo motor may usually be used as a power drive system, and a belt transmission mechanism may usually be used as a power transmission mechanism. Wherein, a belt transmission mechanism may use a toothed belt transmission mechanism or wedged belt transmission mechanism to transmit power. However, considering the noise control of an overall CT scanner system in high speed rotation, a wedged belt transmission mechanism may usually be preferred.

NEUSOFT MEDICAL SYSTEMS CO., LTD. (NMS), founded in 1998 with its world headquarters in China, is a leading supplier of medical equipment, medical IT solutions, and healthcare services. NMS supplies medical equipment with a wide portfolio, including CT, MRI, digital X-ray machine, Ultrasound, PET (Positron Emission Tomography), Linear Accelerator, and Biochemistry Analyser. Currently, NMS' products are exported to over 60 countries and regions around the globe, serving more than 5,000 renowned customers. NMS' latest successful developments, such as the 128 Multi-Slice CT Scanner System, Superconducting MRI, Linear Accelerator, and PET products, have led China to become a global high-end medical equipment producer. As an integrated supplier with extensive experience in large medical equipment, NMS has been committed to the study of avoiding secondary potential harm caused by excessive X-ray irradiation to the subject (e.g., a patient) during the CT scanning process.

### BRIEF DESCRIPTION OF DRAWINGS

Features of the present disclosure are illustrated by way of example and not limited in the following figure(s), in which like numerals indicate like elements, in which:

FIG. 1 is a flowchart illustrating a method for correcting rotation speed of a rotatable gantry in a CT scanner system according to an example of the present disclosure;

FIG. 2 is a flowchart illustrating a method for correcting rotation speed of a rotatable gantry in a CT scanner system according to another example of the present disclosure;

FIG. 3A schematically illustrates a system for correcting rotation speed of a rotatable gantry in a CT scanner system according to an example of the present disclosure;

FIG. 3B is a block diagram illustrating functional modules of the rotation speed correcting mechanism in FIG. 3A according to an example of the present disclosure;

FIG. 4 schematically illustrates a system for correcting rotation speed of a rotatable gantry in a CT scanner system according to another example of the present disclosure;

FIG. 5 schematically illustrates a system for correcting rotation speed of a rotatable gantry in a CT scanner system according to still another example of the present disclosure; and

FIG. 6 schematically illustrates a system for correcting rotation speed of a rotatable gantry in a CT scanner system according to yet another example of the present disclosure.

### DETAILED DESCRIPTION

For simplicity and illustrative purposes, the present disclosure may be described by referring mainly to an example thereof. In the following description, numerous specific details are set forth in order to provide a thorough understanding of the present disclosure. It will be readily apparent however, that the present disclosure may be practiced without limitation to these specific details. In other instances, some methods and structures have not been described in detail so as not to unnecessarily obscure the present disclosure. As used herein, the terms “a” and “an” are intended to denote at least one of a particular element, the term “includes” means includes but not limited to, the term “including” means including but not limited to, and the term “based on” means based at least in part on.

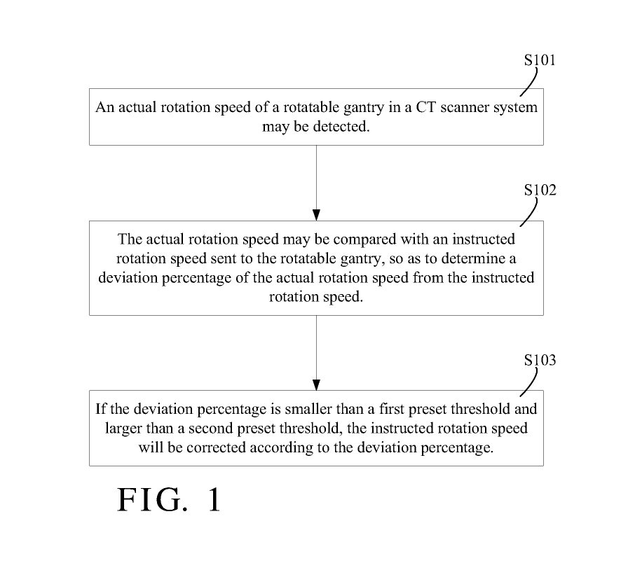

FIG. 1 is a flowchart illustrating a method for correcting rotation speed of a rotatable gantry in a CT scanner system according to an example of the present disclosure, and said method may include blocks S101˜S103.

At block S101, an actual rotation speed of a rotatable gantry in a CT scanner system may be detected.

A method provided in examples of the present disclosure may be applicable to a CT scanner system in which a wedged belt transmission mechanism may be employed as a belt transmission mechanism. Since the transmission ratio of a wedged belt may be depleted in a gradual way over the course of month(s) or even year(s), and sudden changes may be very rare, the time interval between two correcting processes may be adjusted according to practical needs, such as one correcting in one day, or one correcting in one week or one correcting in one month.

At block S102, an actual rotation speed may be compared with an instructed rotation speed sent to a rotatable gantry to determine a deviation percentage of an actual rotation speed from an instructed rotation speed.

Since a wedged belt transmission mechanism may slide at some extent with using time increasing and possible over load, an actual rotation speed may have a deviation from an instructed rotation speed.

According to an example, the deviation percentage of an actual rotation speed from an instructed rotation speed may be calculated by dividing the difference between an actual rotation speed and an instructed rotation speed by an instructed rotation speed.

At block S103, if a deviation percentage is smaller than a first preset threshold and larger than a second preset threshold, the instructed rotation speed will be corrected according to the deviation percentage.

Wherein, the first preset threshold may be a relatively large numerical value such as 10%. When a deviation percentage may be larger than a first preset threshold, it may indicate that the difference between an actual rotation speed and an instructed rotation speed may be too large. In this case, simply correcting rotation speed may not solve the problem and it may request a user to inspect and repair the belt transmission mechanism. A second preset threshold may be smaller than a first preset threshold, and may be a relatively small numerical value such as 0.5%. When the deviation percentage may be smaller than a second preset threshold, it may indicate that the actual rotation speed may be quite close to the instructed rotation speed. In this case, rotation speed correction may not be necessary. Therefore, when the deviation percentage falls between a first preset threshold and a second preset threshold, a rotation speed correction may be required.

When a CT scanner system starts up, the above method may be automatically performed in a preheating process of an X-ray bulb tube in the CT scanner system, without any manual operation, and thereby the operating efficiency may be improved.

According to the above example of the present disclosure, by detecting an actual rotation speed of a rotatable gantry, determining a deviation of an actual rotation speed from an instructed rotation speed, and correcting an instructed rotation speed according to the deviation, a negative impact from the belt transmission mechanism may be decreased as much as possible.

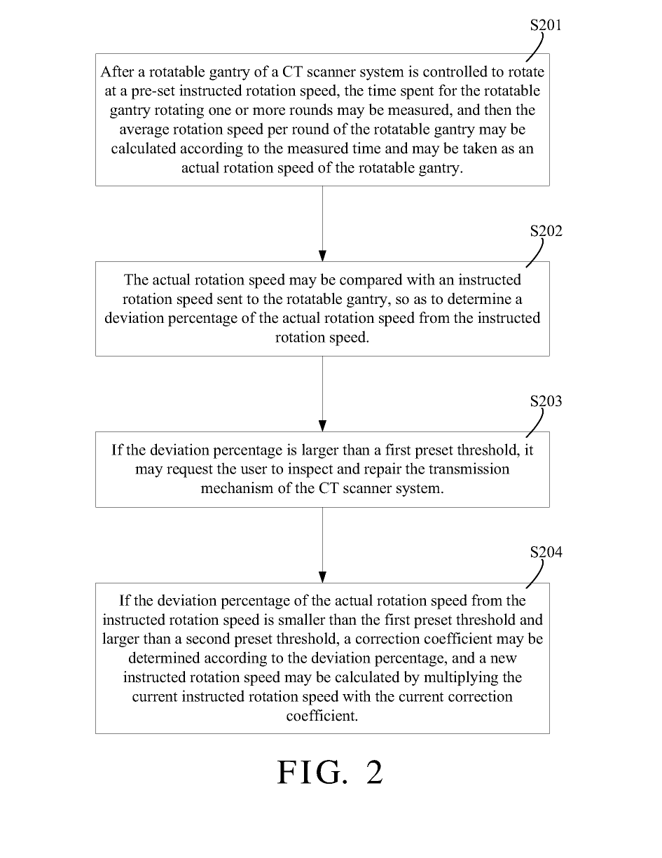

FIG. 2 is a flowchart illustrating a method for correcting rotation speed of a rotatable gantry in a CT scanner system according to another example of the present disclosure, and said method may include the following blocks S201-S204.

At block S201, after a rotatable gantry of a CT scanner system may be controlled to rotate at a preset instructed rotation speed, the time spent for the rotatable gantry rotating one or more rounds may be measured, and then an average rotation speed per round of the rotatable gantry may be calculated according to the measured time and may be taken as an actual rotation speed of the rotatable gantry.

By measuring an average rotation speed per round of a rotatable gantry as the actual rotation speed, an accumulated deviation caused by an unbalanced weight distribution may be avoided, and a delayed response to real time rotation speed control due to a large rotary inertia of the rotatable gantry may also be avoided. Wherein, an unbalanced weight distribution indicates that the weight distribution of a rotatable part in the gantry may not be uniform, and thus the rotation speed per round may have a periodical change.

According to an example of the present disclosure, an impulse counting method may be adopted to measure the time for a rotatable gantry rotating one or more rounds. Since the measured time may be relatively long, an average rotation speed acquired through an impulse counting method may have a high preciseness.

At block S202, an actual rotation speed may be compared with an instructed rotation speed sent to a rotatable gantry so as to determine a deviation percentage of the actual rotation speed from the instructed rotation speed.

According to an example, a deviation percentage of an actual rotation speed from an instructed rotation speed may be calculated by dividing the difference between the actual rotation speed and the instructed rotation speed by the instructed rotation speed.

At block S203, if the deviation percentage of an actual rotation speed from an instructed rotation speed may be larger than a first preset threshold, it may request the user to inspect and repair a transmission mechanism of the CT scanner system.

Wherein, a first preset threshold may be a relatively large numerical value such as 10%. And when the deviation percentage may be larger than a first threshold value, it may indicate that the actual rotation speed deviated from the instructed rotation speed too much. In this case, simply correcting rotation speed may not solve the problem and it may request a user to inspect and repair a belt transmission mechanism of the CT scanner system.

It should be noted that, block S203 and the subsequent block S204 may not be defined sequentially and may be executed in an order different from that mentioned above.

At block S204, if a deviation percentage of an actual rotation speed from an instructed rotation speed is smaller than a first preset threshold and larger than a second preset threshold, a correction coefficient may be determined according to the deviation percentage obtained, and a new instructed rotation speed may be calculated by multiplying a current instructed rotation speed with a current correction coefficient.

According to an example, different deviation percentages may correspond to different correction coefficients, so as to ensure that a larger deviation in a rotation speed may be corrected much more. For example, a correction coefficient may be set as proportional to a deviation percentage, thus a larger deviation percentage may correspond to a larger correction coefficient.

According to the above example of the present disclosure, since a new instructed rotation speed may be the product of a correction coefficient with the instructed rotation speed, a new instructed rotation speed may be considered as having been corrected properly such that a deviation in rotation speed due to a belt transmission mechanism may be decreased.

According to another example of the present disclosure, a system for correcting rotation speed of a rotatable gantry in a CT scanner system may be provided, and its operating principle will be illustrated in detail with reference to the drawings.

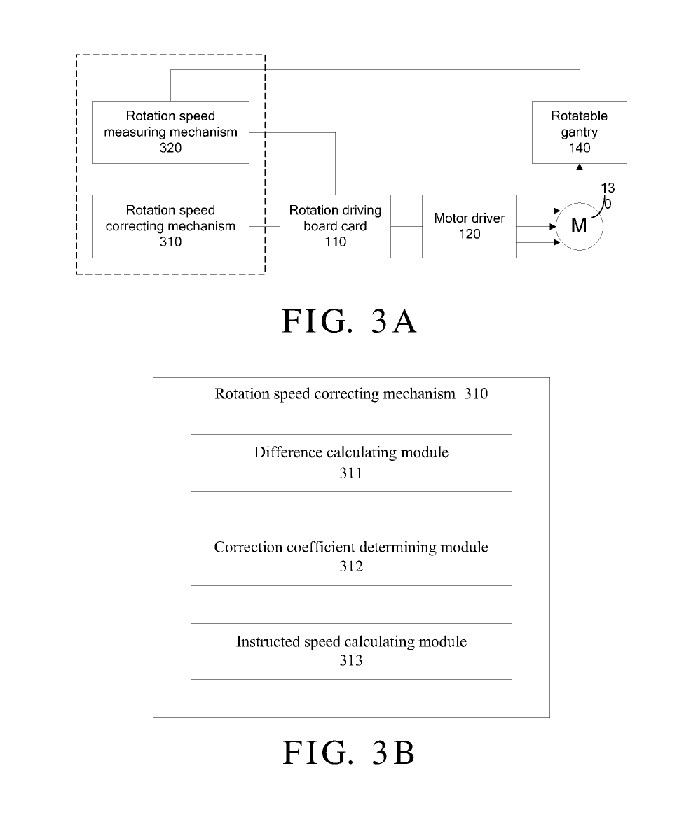

FIG. 3A schematically illustrates a system for correcting rotation speed of a rotatable gantry in a CT scanner system according to an example of the present disclosure. As shown in FIG. 3A, a system for correcting rotation speed may be applied to a CT scanner system including a rotation driving board card 110, a motor driver 120, a motor 130 and a rotatable gantry 140, and may include a rotation speed correcting mechanism 310 and a rotation speed measuring mechanism 320.

Wherein, a rotation driving board card 110 may be configured to control the rotation of a rotatable gantry 140 by sending a rotation control command to the motor driver 120. According to an example, a rotation driving board card 110 may generate a rotation control command according to an instructed rotation speed from the rotation speed correcting mechanism 310. Further, a rotation driving board card 110 may be provided with a non-volatile memory configured to store data about rotation speed correction.

A motor driver 120 may be configured to drive a motor 130. According to an example, a rotation driving board card 110 may send a rotation control command to a motor driver 120 through a standard differential communication interface.

The motor 130 may be configured to drive a rotatable gantry 140. For example, a motor 130 may drive the rotatable gantry 140 by using a wedged belt to transmit power.

The rotatable gantry 140, as a rotation part of a gantry in the CT scanner system, may usually be provided with an X-ray bulb tube for emitting X-ray beam.

The rotation speed measuring mechanism 320 may be positioned onto a rotatable gantry 140 of a CT scanner system, and configured to measure the average rotation speed per round of a rotatable gantry 140. According to an example, a rotation speed measuring mechanism 320 may communicate with a rotation driving board card 110 through a standard differential communication interface, such as transmitting an average rotation speed per round of a rotatable gantry 140 to a rotation driving board card 110 through a standard differential communication interface.

According to an example, a rotation speed measuring mechanism 320 may comprise an impulse generator configured to generate one impulse once a rotatable gantry 140 rotates one round. Thus, by counting the impulses generated by an impulse generator, the time spent for a rotatable gantry 140 rotating one or more rounds may be acquired. For example, the time between two adjacent impulses may be equal to the time spent for the rotatable gantry 140 rotating one round, and the time between the i^{th} impulse and the (i+N)^{th} impulse may be equal to the time spent for the rotatable gantry 140 rotating N rounds. Then, an average rotation speed per round of a rotatable gantry 140 may be calculated based on the acquired time.

As illustrated in FIG. 3A, a rotation speed measuring mechanism 320 may send a measured average rotation speed to a rotation driving board card 110 as an actual rotation speed of a rotatable gantry 140, and then a rotation driving board card 110 may send an actual rotation speed of the rotatable gantry 140 to a rotation speed correcting mechanism 310. According to an example, the rotation driving board card 110 and the rotation speed correcting mechanism 310 may communicate with each other through a Personal Computer (PC) bus.

In this way, after acquiring an instructed rotation speed and an actual rotation speed, a rotation speed correcting mechanism 310 may correct rotation speed based on a deviation percentage of the actual rotation speed from the instructed rotation speed, and send a corrected instructed rotation speed to a rotation driving board card 110.

For example, if a deviation percentage of an actual rotation speed from an instructed rotation speed may be smaller than a first preset threshold and larger than a second preset threshold, an instructed rotation speed may be corrected according to a deviation percentage. Wherein, a first preset threshold may be a relatively large numerical value, and when a deviation percentage may be above a preset threshold value, it may indicate that the actual rotation speed deviated from the instructed rotation speed too much. In this case, simply correcting the rotation speed may not solve the problem and a user may be requested to inspect and repair a belt transmission mechanism of the CT scanner system. The second preset threshold may be a relatively small numerical value and may be smaller than a first preset threshold. For example, a first preset threshold may be set as 10%, and a second preset threshold may be set as 0.5%, etc.

According to an example, an instructed rotation speed may be corrected by: acquiring a correction coefficient corresponding to a deviation percentage, and calculating a new instructed rotation speed by multiplying a correction coefficient with a current instructed rotation speed. Wherein, a correction coefficient may be stored in the rotation driving board card 110 or the rotation speed correcting mechanism 310.

According to a specific example, different deviation percentages may correspond to different correction coefficients so as to guarantee that a larger deviation in a rotation speed may be corrected much more. For example, a correction coefficient may be set as proportional to a deviation percentage, and a larger deviation percentage may correspond to a larger correction coefficient. Or, a deviation percentage may be acquired by dividing the difference between an actual rotation speed and an instructed rotation speed by an instructed rotation speed, and a correction coefficient may be acquired by dividing the instructed rotation speed by the actual rotation speed.

It should be noted that, when a CT scanner system starts up, the present system may automatically correct rotation speed of a rotatable gantry in the CT scanner system during a preheating process of an X-ray bulb tube in the CT system, without any manual operation, and thereby the efficiency of operation may be improved.

According to an example, as shown in FIG. 3B, a rotation speed correcting mechanism 310 may include:

A difference calculating module 311, may be configured to calculate a deviation percentage of the actual rotation speed from the instructed rotation speed;

A correction coefficient determining module 312, may be configured to acquire a correction coefficient according to the deviation percentage; and

An instructed speed calculating module 313, may be configured to calculate a new instructed rotation speed by multiplying the correction coefficient with the current instructed rotation speed.

Generally, a CT scanner system may include a CT scanner controller configured to control the overall operation of the CT scanner system, such as sending a first instructed rotation speed. The CT scanner controller may be a console computer operated by a user. For example, a CT scanner controller may be installed with CT scanner control software.

Generally, a CT scanner system may further include a gantry controller configured to control the motion of a rotatable gantry 140 in the CT scanner system, such as sending an instructed rotation speed to a rotation driving board card 110. A gantry controller may be a control computer installed onto a rotatable gantry 140 of the CT scanner system. According to an example, a gantry controller may communicate with a rotation driving board card 110 of the CT scanner system through a PC bus, and/or, a gantry controller may communicate with the CT scanner controller through network cable.

According to some examples, a rotation speed correcting mechanism 310 may be implemented with a CT scanner controller and/or the gantry controller.

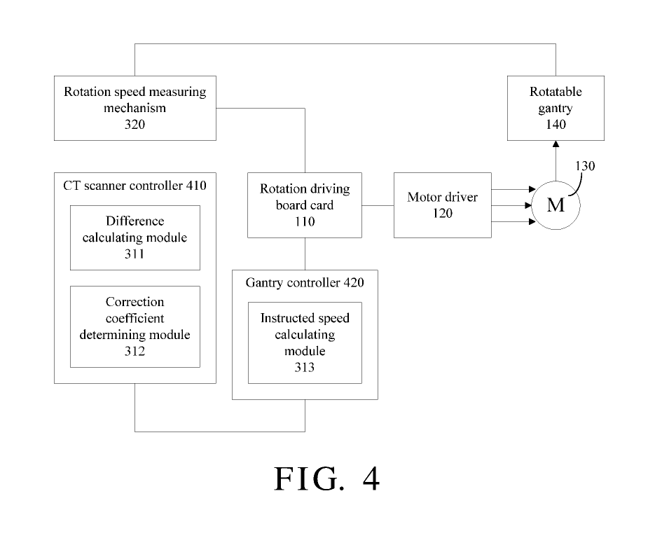

For example, as illustrated in FIG. 4, a difference calculating module 311 and a correction coefficient determining module 312 in a rotation speed correcting mechanism 310 may be implemented in the CT scanner controller 410, and an instructed speed calculating module 313 in the rotation speed correcting mechanism 310 may be implemented in the gantry controller 420.

In this case, a process for controlling rotation speed of a rotatable gantry 140 in a CT scanner system may include:

At block A1, when the CT scanner system is powered up, a CT scanner controller 410 starts preparation work for the CT scanner system, including sending a preset instructed rotation speed such as 1S/R to a gantry controller 420 through network cable;

At block A2, after receiving an instructed rotation speed from a CT scanner controller 410, a gantry controller 420 sends an instructed rotation speed such as 1S/R without any correction coefficient to a rotation driving board card 110 through PC bus;

At block A3, when receiving the instructed rotation speed, a rotation driving board card 110 generates a rotation speed control command and sends a rotation speed control command to a motor driver 120 through differential bus;

At block A4, a motor driver 120 controls a motor 130 to rotate according to a received rotation speed control command;

At block A5, the motor 130 rotates under a control from a motor driver 120, and the rotation of a motor 130 is transmitted to a rotatable part of a gantry in the CT scanner system (i.e., the rotatable gantry 140) through power transmission of wedged belt, so as to drive the rotatable gantry 140 to rotate;

At block A6, a rotation speed measuring mechanism 320 on a gantry may measure an average rotation speed per round of a rotatable gantry 140, and send an average rotation speed to a rotation driving board card 110 through a standard differential communication interface;

At block A7, when receiving an average rotation speed from a rotation speed measuring mechanism 310, a rotation driving board card 110 takes an average rotation speed as an actual rotation speed of a rotatable gantry 140 and sends it to a gantry controller 420 through a PC bus, and a gantry controller 420 may further send an actual rotation speed to a CT scanner controller 410 through network cable;

At block A8, a CT scanner controller 410 calculates a deviation percentage of the feed backed actual rotation speed from the preset instructed rotation speed. If a deviation percentage is larger than a first preset threshold, it may indicate a user shall be requested to inspect and repair a transmission mechanism of the CT scanner system. If a deviation percentage is smaller than a first preset threshold and larger than a second preset threshold, it may indicate that an instructed rotation speed shall be corrected and a correction coefficient may be determined accordingly;

At block A9, a CT scanner controller 410 sends a correction coefficient to a gantry controller 420 through network cable, and a gantry controller 420 may store a correction coefficient into itself or write it into a non-volatile memory of a rotation driving board card 110 through PC bus;

At block A10, a gantry controller 420 calculates a new instructed rotation speed by multiplying a current instructed rotation speed with a correction coefficient, and sends a new instructed rotation speed to a rotation driving board card 110 through PC bus;

At block A11, a rotation driving board card 110 performs control according to a new instructed rotation speed, such that the process returns to above block A3;

In the present example, a rotation speed correction may be mainly performed by a gantry controller 420, and a CT scanner controller may be relatively independent to the process for controlling rotation speed of the rotatable gantry in the CT scanner system.

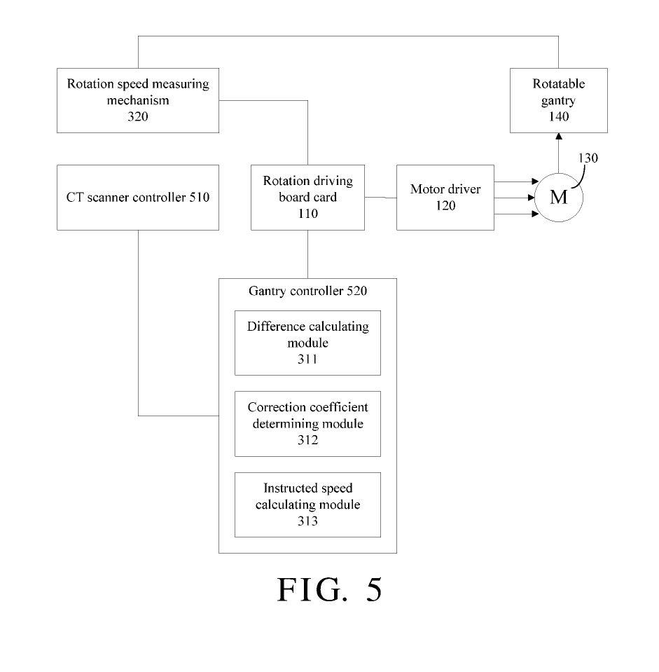

According to another example of the present disclosure, as illustrated in FIG. 5, a difference calculating module 311, a correction coefficient determining module 312 and an instructed speed calculating module 313 in a rotation speed correcting mechanism 310 may all be implemented in a gantry controller 520.

In this case, a process for controlling rotation speed of the rotatable gantry 140 in the CT scanner system may include:

At block B1, the above blocks A1-A7 may be performed;

At block B2, a gantry controller 520 calculates a deviation percentage of a provided actual rotation speed from a current instructed rotation speed. If a deviation percentage is larger than a first preset threshold, a gantry controller 520 may transmit the information to a CT scanner controller 510, such that the CT scanner controller 510 may indicate a user to inspect and repair a transmission mechanism of the CT scanner system. If a deviation percentage is smaller than a first preset threshold and larger than a second preset threshold, a gantry controller 520 may determine a correction coefficient according to a deviation percentage;

At block B3, a gantry controller 520 stores a correction coefficient into itself, or writes it into a non-volatile memory of a rotation driving board card 110 through PC bus;

At block B4, a gantry controller 520 calculates a new instructed rotation speed by multiplying a current instructed rotation speed with a correction coefficient, and then sends a new instructed rotation speed to a rotation driving board card 110 through PC bus;

At block B5, a rotation driving board card 110 performs control according to a new instructed rotation speed, such that the process returns to above block A3.

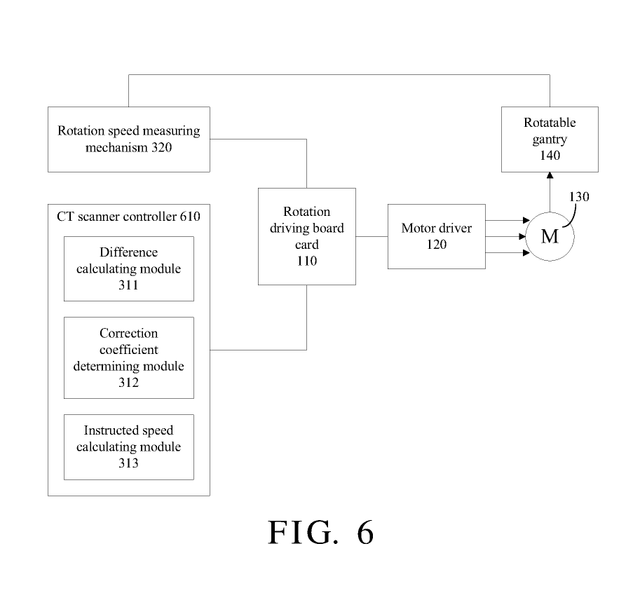

According to still another example of the present disclosure, as illustrated in FIG. 6, a difference calculating module 311, a correction coefficient determining module 312 and an instructed speed calculating module 313 in a rotation speed correcting mechanism 310 may all be implemented in the CT scanner controller 610.

In this case, a process for controlling rotation speed of the rotatable gantry 140 for the CT scanner system may include: when a CT scanner system is powered up, the CT scanner controller 610 reads a correction coefficient pre-stored in itself or in a rotation driving board card 110, and directly sends an instructed rotation speed calculated by multiplying the correction coefficient with a preset instructed rotation speed to a rotation driving board card 110.

The above may be preferred examples of the present disclosure and are not intended to limit the disclosure within the spirit and principles of the present disclosure, any changes made, equivalent replacement, or improvement in the protection of the present disclosure should contain within the range.

The methods, processes and units described herein may be implemented by hardware (including hardware logic circuitry), software or firmware or a combination thereof. The term ‘processor’ may be interpreted broadly to include a processing unit, ASIC, logic unit, or programmable gate array etc. The processes, methods and functional units may all be performed by the one or more processors; reference in this disclosure or the claims to a ‘processor’ should thus be interpreted to mean ‘one or more processors’.

Further, the processes, methods and functional units described in this disclosure may be implemented in the form of a computer software product. The computer software product may be stored in a storage medium and comprises a plurality of instructions for making a processor to implement the methods recited in the examples of the present disclosure.

The figures are only illustrations of an example, wherein the units or procedure shown in the figures are not necessarily essential for implementing the present disclosure. Those skilled in the art will understand that the units in the device in the example may be arranged in the device in the examples as described, or may be alternatively located in one or more devices different from that in the examples. The units in the examples described may be combined into one module or further divided into a plurality of sub-units.

Although the flowcharts described show a specific order of execution, the order of execution may differ from that which may be depicted. For example, the order of execution of two or more blocks may be changed relative to the order shown. Also, two or more blocks shown in succession may be executed concurrently or with partial concurrence. All such variations may be within the scope of the present disclosure.

Throughout the present disclosure, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for correcting rotation speed of a rotatable gantry in a CT scanner system, the method comprising:
detecting an actual rotation speed of the rotatable gantry;
comparing the actual rotation speed with an instructed rotation speed sent to the rotatable gantry so as to determine a deviation percentage of the actual rotation speed from the instructed rotation speed; and
if the deviation percentage of the actual rotation speed from the instructed rotation speed is smaller than a first preset threshold and larger than a second preset threshold, correcting the instructed rotation speed according to the deviation percentage.

2. The method of claim 1, wherein, said detecting an actual rotation speed of the rotatable gantry, includes:
controlling the rotatable gantry to rotate at a preset instructed rotation speed;
measuring the time spent for the rotatable gantry rotating one or more rounds; and
calculating an average rotation speed per round of the rotatable gantry based on the measured time as the actual rotation speed.

3. The method of claim 1, wherein, said correcting the instructed rotation speed according to the deviation percentage, includes:
determining a correction coefficient according to the deviation percentage; and
calculating a new instructed rotation speed by multiplying the correction coefficient with the current instructed rotation speed.

4. The method of claim 1, further including:
if the deviation percentage of the actual rotation speed from the instructed rotation speed is larger than the first preset threshold, indicating the user to inspect the transmission mechanism of the CT scanner system.

5. A system for correcting rotation speed of a rotatable gantry in a CT scanner system, including:
a rotation speed measuring mechanism, which is positioned on the rotatable gantry and configured to measure an actual rotation speed of the rotatable gantry, and send the actual rotation speed to a rotation driving board card of the CT scanner system; and
a rotation speed correcting mechanism, which is configured to receive the actual rotation speed from the rotation driving board card, compare the actual rotation speed with an instructed rotation speed sent to the rotatable gantry and determine a deviation percentage of the actual rotation speed from the instructed rotation speed;
wherein if the deviation percentage of the actual rotation speed from the instructed rotation speed is smaller than a first preset threshold and larger than a second preset threshold, the rotation speed correcting mechanism corrects the instructed rotation speed according to the deviation percentage.

6. The system of claim 5, wherein, the rotation speed measuring mechanism includes:
an impulse generator, which is configured to generate one impulse once the rotatable gantry rotates one round; and
a speed calculator, which is configured to determine the time spent for the rotatable gantry rotating one or more rounds based on the impulse signal output from the impulse generator, and calculate an average rotation speed per round of the rotatable gantry based on the determined time as the actual rotation speed.

7. The system of claim 5, wherein, the rotation speed correcting mechanism includes:
a difference calculating module, which is configured to calculate the deviation percentage of the actual rotation speed from the instructed rotation speed;
a correction coefficient determining module, which is configured to determine a correction coefficient according to the deviation percentage; and
an instructed speed calculating module, which is configured to calculate a new instructed rotation speed by multiplying the correction coefficient with the current instructed rotation speed.

8. The system of claim 7, wherein,
the difference calculating module is implemented in a CT scanner controller of the CT scanner system, the CT scanner controller is configured to control overall operation of the CT scanner system;
the correction coefficient determining module is implemented in the CT scanner controller; and
the instructed speed calculating module is implemented in the CT scanner controller.

9. The system of claim 8, wherein, the CT scanner controller communicates with the rotation driving board card through network cable.

10. The system of claim 8, wherein, the correction efficient is stored in the CT scanner controller or the rotation driving board card.

11. The system of claim 7, wherein,
the difference calculating module is implemented in a CT scanner controller of the CT scanner system, the CT scanner controller is configured to control overall operation of the CT scanner system including sending an initial instructed rotation speed of the rotatable gantry;
the correction coefficient determining module is implemented in the CT scanner controller; and
the instructed speed calculating module is implemented in a gantry controller of the CT scanner system, the gantry controller is configured to control the motion of the rotatable gantry.

12. The system of claim 11, wherein,
the CT scanner controller communicates with the gantry controller through network cable; and
the gantry controller communicates with the rotation driving board card through PC bus.

13. The system of claim 11, wherein, the correction coefficient is stored in the gantry controller or the rotation driving board card.

14. The system of claim 7, wherein,
the difference calculating module is implemented in a gantry controller of the CT scanner system, the gantry controller is configured to control motion of the rotatable gantry;
the correction coefficient determining module is implemented in the gantry controller; and
the instructed speed calculating module is implemented in the gantry controller.

15. The system of claim 14, wherein,
the gantry controller communicates with a CT scanner controller of the CT scanner system through network cable, the CT scanner controller is configured to control overall operation of the CT scanner system including sending an initial instructed rotation speed of the rotatable gantry; and
the gantry controller communicates with the rotation driving board card through PC bus.

16. The system of claim 14, wherein, the correction coefficient is stored in the gantry controller or the rotation driving board card.
